(19) United States
(12) Patent Application Publication
(10) Pub. No.: US 2004/0204735 A1
(43) Pub. Date: Oct. 14, 2004

(54) Subcutaneous dissection tool incorporating pharmacological agent delivery

(76) Inventors: Jason Alan Shiroff, Shoreview, MN (US); Curtis Charles Lindstrom, Roseville, MN (US); Adam W. Cates, Minneapolis, MN (US)

Correspondence Address: Crawford Maunu PLLC, Suite 390, 1270 Northland Drive, St. Paul, MN, 55120, US

(21) Appl. No.: 10/625,833

(22) Filed: Jul. 23, 2003

Related U.S. Application Data
(60) Provisional application No. 60/462,272, filed on Apr. 11, 2003.

(51) Int. Cl. A61B 17/00

(52) U.S. Cl. 606/190

(57) ABSTRACT

Tissue dissection instruments and methods provide for fluid delivery and aspiration during subcutaneous dissection. A dissection tool includes a handle and a dissecting member. The dissecting member extends from the distal end of the handle, and a fluid channel system extends from at least the proximal end to the distal end of the dissecting member. The fluid channel system terminates in a port system. The port system may include one or more apertures, one or more channels, and be adapted to provide fluid transport and/or aspiration, such fluids including irrigation fluids, fluids having analgesics, antibiotics, hemostatic agents, healing accelerating agents, agents that improve the electrical properties of tissue, and combinations of fluids and agents. The dissection tool may be straight or curved, rigid or malleable, and shaped to provide dissection paths suitable for the implantation of subcutaneous electrodes.

### RELATED APPLICATIONS

This application claims the benefit of Provisional Patent Application Serial No. 60/462,272, filed on Apr. 11, 2003, to which priority is claimed pursuant to 35 U.S.C. §119 (e) and which is hereby incorporated herein by reference.

The present invention relates generally to tissue dissection instruments and, more particularly, to subcutaneous tissue dissection instruments incorporating pharmacological agent delivery.

### BACKGROUND OF THE INVENTION

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses that are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating the arrhythmias.

Implantable cardioverter/defibrillators (ICDs) have been used as an effective treatment for patients with serious cardiac arrhythmias. For example, a typical ICD includes one or more endocardial leads to which at least one defibrillation electrode is connected. Such ICDs are capable of delivering high-energy shocks to the heart, interrupting the ventricular tachyarrythmia or ventricular fibrillation, and allowing the heart to resume normal sinus rhythm. ICDs may also include pacing functionality.

Although ICDs are very effective at preventing Sudden Cardiac Death (SCD), most people at risk of SCD are not provided with implantable defibrillators. The primary reasons for this unfortunate reality include the limited number of physicians qualified to perform transvenous lead/electrode implantation, a limited number of surgical facilities adequately equipped to accommodate such cardiac procedures, and a limited number of the at-risk patient population that can safely undergo the required endocardial or epicardial lead/electrode implant procedure.

For reasons stated above, and for other reasons which will become apparent to those skilled in the art upon reading the present specification, there is a need for systems and methods that provide for sensing cardiac activity and delivering defibrillation and/or pacing therapies without the need for endocardial or epicardial leads/electrodes. There is a particular need for tools and techniques that facilitate implantation of such systems. The present invention fulfills these and other needs, and addresses deficiencies in known systems and techniques.

### SUMMARY OF THE INVENTION

The present invention is directed to subcutaneous dissection tools, methods and systems that, in general, provide access for deployment of subcutaneous electrodes, cans, and housings used in transthoracic defibrillation therapies, cardiac monitoring systems, transthoracic pacing therapies, or a combination of the above. Embodiments of the present invention include subcutaneous dissection tools, systems, and kits that include pharmacological agent delivery during dissection.

According to one embodiment, a dissection tool of the present invention includes a handle having a proximal end and a distal end, and an elongated dissecting member having a proximal end and a distal end. The elongated dissecting member extends from the distal end of the handle. A fluid channel system extends from at least the proximal end of the elongated dissecting member to the distal end of the elongated dissecting member.

The fluid channel system may terminate in a port system. The port system may include one or more apertures, one or more channels, and be adapted to provide fluid transport and/or aspiration, such fluids including, for example, irrigation fluids, fluids having analgesics, antibiotics, hemostatic agents, healing accelerating agents, agents that improve the electrical properties of tissue, and combinations of fluids and agents. The fluid dispensed through the fluid channel system is typically a liquid, but may alternatively be a gas.

In alternate embodiments, the apertures of the port system may have associated valves such as, for example, aperture covers to keep debris out of the fluid channels. Another embodiment includes a sheath removably surrounding the dissector. The sheath may be left in place after removal of the dissector to facilitate electrode delivery and implantation. The sheath may thereafter be stripped out of the dissection path to fix the electrode lead.

The dissection tool may be straight or curved, rigid or malleable, and shaped to provide dissection paths suitable for the implantation of subcutaneous electrodes. For example, the dissection tool may have a single curved portion defined by a single radius or a complex curvature defined by two or more radii. A system incorporating dissection tools in accordance with the present invention may include fluid storage, a pump, and tubing for fluid delivery.

According to another embodiment, a dissection tool of the present invention includes a lead lumen. The lead lumen extends between the proximal and distal ends of the dissection tool and is configured to receive a lead, such as a pacing lead, a defibrillation lead or a sensor lead. The lead lumen is typically provided as a lumen distinct from one or more fluid delivery channels/lumens incorporated into the dissection tool. It is understood that a dissection tool employing a lead lumen need not incorporate a fluid delivery capability.

Another embodiment of the present invention provides a method of dissection. The method of dissecting subcutaneous tissue in accordance with the present invention includes providing a dissection tool with a pharmacological agent delivery channel, dissecting subcutaneous tissue with the dissection tool, and delivering a fluid from the dissection tool during dissection. The fluid delivered may include agents that provide analgesia, hemostasis, bacterial fighting and infection fighting, increased tissue healing, flush out debris, and improve electrical properties of tissue. The dissection method may include steps of following the subcutaneous plane for dissection along the curvature of the rib cage, for example.

A further embodiment of the present invention provides methods of dissection using a curved or malleable dissector particularly suited to dissect a path for subcutaneous electrode placement. Yet another embodiment of the present invention is directed to kits that include selected tools, implements, and devices for performing subcutaneous dissection including fluid delivery.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIGS. 1A and 1B are views of a transthoracic cardiac monitoring and/or stimulation device as implanted in a patient;

FIG. 2A is a perspective view of a subcutaneous dissection system in accordance with the present invention;

FIG. 2B is a perspective view of a another subcutaneous dissection system in accordance with the present invention;

FIG. 3 is a magnified perspective view of the distal end of the dissector illustrated in FIGS. 2A and 2B;

FIG. 4 is a magnified sectional view bisecting the distal end illustrated in FIG. 3;

FIG. 5 is a section view of the elongated dissecting member having a surrounding sheath;

FIG. 6 is a plan view of the distal end of a dissector including a port system having aperture covers;

FIG. 7 is a magnified transparent perspective view of the distal end of a dissector having a central lead lumen in accordance with the present invention;

FIG. 8 is a magnified plan view of the dissector illustrated in FIG. 7;

FIG. 9 is a magnified transparent perspective view of the distal end of a dissector having a central lead lumen and manifold in accordance with the present invention; and

FIG. 10 is a magnified plan view of the dissector illustrated in FIG. 9.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

A device in accordance with the present invention can include one or more of the features, structures, methods, or combinations thereof described herein below. For example, a subcutaneous dissector or dissection method can be implemented to include one or more of the advantageous features and/or processes described below. It is intended that such a dissection device or method need not include all of the features and functions described herein, but can be implemented to include selected features and functions that provide for unique structures and/or functionality.

In general terms, a dissection tool of the present invention can be used to facilitate implantation of a subcutaneous cardiac monitoring and/or stimulation device. One such device is an implantable transthoracic cardiac sensing and/or stimulation (ITCS) device that can be implanted under the skin in the chest region of a patient. The ITCS device may, for example, be implanted subcutaneously such that all or selected elements of the device are positioned on the patient's front, back, side, or other body locations suitable for sensing cardiac activity and delivering cardiac stimulation therapy. It is understood that elements of the ITCS device may be located at several different body locations, such as in the chest, abdominal, or subclavian region with electrode elements respectively positioned at different regions near, around, in, or on the heart. A dissection tool and methodology of the present invention can be used to provide electrode and device access at various subcutaneous body locations.

The primary housing (e.g., the active or non-active can) of the ITCS device, for example, can be configured for positioning outside of the rib cage at an intercostal or subcostal location, within the abdomen, or in the upper chest region (e.g., subclavian location, such as above the third rib). In one implementation, one or more electrodes can be located on the primary housing and/or at other locations about, but not in direct contact with the heart, great vessel or coronary vasculature. In another implementation, one or more electrodes can be located in direct contact with the heart, great vessel or coronary vasculature, such as via one or more leads implanted by use of conventional transvenous delivery approaches. In another implementation, for example, one or more subcutaneous electrode subsystems or electrode arrays can be used to sense cardiac activity and deliver cardiac stimulation energy in an ITCS device configuration employing an active can or a configuration employing a non-active can. Electrodes can be situated at anterior and/or posterior locations relative to the heart.

Due to the number of combinations of electrodes and ITCS devices, and the variability of anatomy and the presentation of conditions amongst patients, surgical kits are often assembled prior to surgery to provide the basic combinations of devices, leads, and ancillary components necessary to perform the surgical procedure. As will be discussed in detail below, dissection kits of the present invention can be assembled to include one or more dissection tools, including those that provide for fluid delivery, one or more electrodes and leads, one or more cans or housings, and combinations of these and other subcutaneous components. For example, kits can include an assortment of dissection tools of various sizes, shapes, and lengths. The dissection tools can be unitary or separable. For example, a common handle section can be configured to accommodate a number of different elongated dissecting members selectable by the clinician.

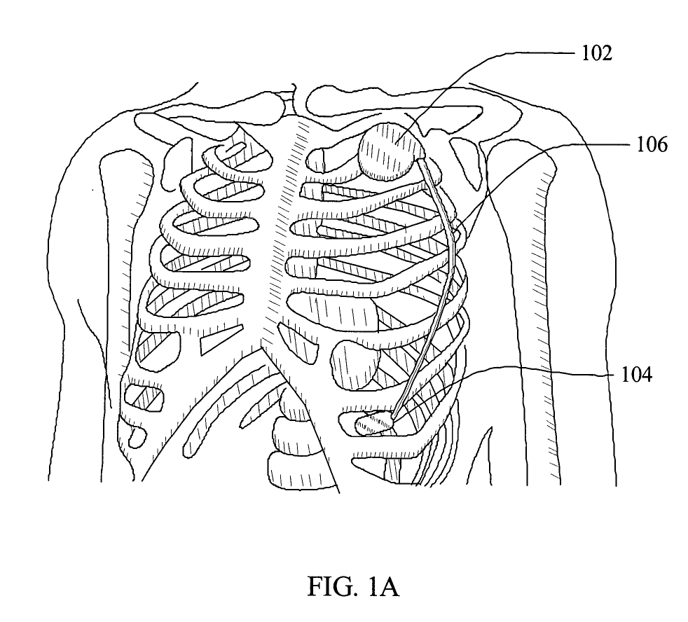

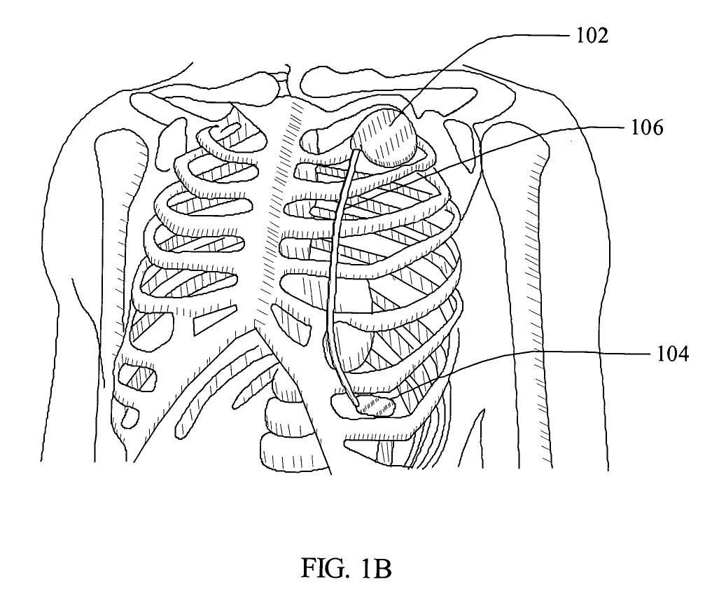

Referring now to FIGS. 1A and 1B of the drawings, there is shown a configuration of a transthoracic cardiac sensing and/or stimulation (ITCS) device implanted in the chest region of a patient at different locations by use of a dissection tool of the present invention. In the particular configuration shown in FIGS. 1A and 1B, the ITCS device includes a housing 102 within which various cardiac sensing, detection, processing, and energy delivery circuitry can be housed. The housing 102 is typically configured to include one or more electrodes (e.g., can electrode and/or indifferent electrode). Although the housing 102 is typically configured as an active can, it is appreciated that a non-active can configuration may be implemented, in which case at least two electrodes spaced apart from the housing 102 are employed. An ITCS system according to this approach is distinct from conventional approaches in that it is preferably configured to include a combination of two or more electrode subsystems that are implanted subcutaneously in the anterior thorax.

In the configuration shown in FIGS. 1A and 1B, a subcutaneous electrode 104 can be positioned under the skin in the chest region and situated distal from the housing 102. The subcutaneous and, if applicable, housing electrode(s) can be positioned about the heart at various locations and orientations, such as at various anterior and/or posterior locations relative to the heart. The subcutaneous electrode 104 is electrically coupled to circuitry within the housing 102 via a lead assembly 106. One or more conductors (e.g., coils or cables) are provided within the lead assembly 106 and electrically couple the subcutaneous electrode 104 with circuitry in the housing 102. One or more sense, sense/pace or defibrillation electrodes can be situated on the elongated structure of the electrode support, the housing 102, and/or the distal electrode assembly (shown as subcutaneous electrode 104 in the configuration shown in FIGS. 1A and 1B).

In one configuration, the lead assembly 106 is generally flexible and has a construction similar to conventional implantable, medical electrical leads (e.g., defibrillation leads or combined defibrillation/pacing leads). In another configuration, the lead assembly 106 is constructed to be somewhat flexible, yet has an elastic, spring, or mechanical memory that retains a desired configuration after being shaped or manipulated by a clinician. For example, the lead assembly 106 can incorporate a gooseneck or braid system that can be distorted under manual force to take on a desired shape. In this manner, the lead assembly 106 can be shape-fit to accommodate the unique anatomical configuration of a given patient, and generally retains a customized shape after implantation. Shaping of the lead assembly 106 according to this configuration can occur prior to, and during, ITCS device implantation.

In accordance with a further configuration, the lead assembly 106 includes a rigid electrode support assembly, such as a rigid elongated structure that positionally stabilizes the subcutaneous electrode 104 with respect to the housing 102. In this configuration, the rigidity of the elongated structure maintains a desired spacing between the subcutaneous electrode 104 and the housing 102, and a desired orientation of the subcutaneous electrode 104/housing 102 relative to the patient's heart. The elongated structure can be formed from a structural plastic, composite or metallic material, and comprises, or is covered by, a biocompatible material. Appropriate electrical isolation between the housing 102 and the subcutaneous electrode 104 is provided in cases where the elongated structure is formed from an electrically conductive material, such as metal.

In one configuration, the rigid electrode support assembly and the housing 102 define a unitary structure (i.e., a single housing/unit). The electronic components and electrode conductors/connectors are disposed within or on the unitary ITCS device housing/electrode support assembly. At least two electrodes are supported on the unitary structure near opposing ends of the housing/electrode support assembly. The unitary structure can have an arcuate or angled shape, for example.

According to another configuration, the rigid electrode support assembly defines a physically separable unit relative to the housing 102. The rigid electrode support assembly includes mechanical and electrical couplings that facilitate mating engagement with corresponding mechanical and electrical couplings of the housing 102. For example, a header block arrangement can be configured to include both electrical and mechanical couplings that provide for mechanical and electrical connections between the rigid electrode support assembly and housing 102. The header block arrangement can be provided on the housing 102 or the rigid electrode support assembly. Alternatively, a mechanical/electrical coupler can be used to establish mechanical and electrical connections between the rigid electrode support assembly and the housing 102. In such a configuration, a variety of different electrode support assemblies of varying shapes, sizes, and electrode configurations can be made available for physically and electrically connecting to a standard ITCS device.

It is noted that the electrodes and the lead assembly 106 can be configured to assume a variety of shapes. For example, the lead assembly 106 can have a wedge, chevron, flattened oval, or a ribbon shape, and the subcutaneous electrode 104 can comprise a number of spaced electrodes, such as an array or band of electrodes. Moreover, two or more subcutaneous electrodes 104 can be mounted to multiple electrode support assemblies 106 to achieve a desired spaced relationship amongst the subcutaneous electrodes 104.

Accordingly, dissection tools of the present invention can be shaped to provide appropriate access for specific electrodes or families of electrodes, electrode support assemblies, and/or leads. For example, a dissection tool of the present invention may be adapted to provide a chevron shaped tunnel, possibly having a particular radius of curvature, in order to facilitate placement of a semi-rigid chevron shaped curved electrode. Likewise, a kit may be assembled having particular shaped electrodes along with particular dissectors adapted for placement of the specific electrodes. The physician can use a number of specifically shaped dissection tools during an implant procedure.

Depending on the configuration of a particular ITCS device, a delivery system incorporating drug/fluid delivery can advantageously be used to facilitate proper placement and orientation of the ITCS device housing and subcutaneous electrode(s). According to one configuration of such a delivery system, a long metal rod similar to conventional trocars can be used to perform small diameter blunt tissue dissection of the subdermal layers. This tool may be pre-formed to assume a straight or curved shape to facilitate placement of the subcutaneous electrode, or it may be sufficiently flexible to allow the physician to shape it appropriately for a given patient.

Exemplary delivery tools, aspects of which can be incorporated into an ITCS device delivery tool in accordance with the present invention, are disclosed in commonly owned U.S. Pat. No. 5,300,106 and U.S. Patent Application entitled “Tunneling Tool with Subcutaneous Transdermal Illumination,” filed concurrently herewith under Attorney Docket No. GUID.619PA, which are hereby incorporated herein by reference. These and other conventional delivery devices can advantageously be modified to incorporate a drug/fluid delivery capability and other structural and functional features as described herein. An improved ITCS device delivery tool in accordance with the present invention is described below.

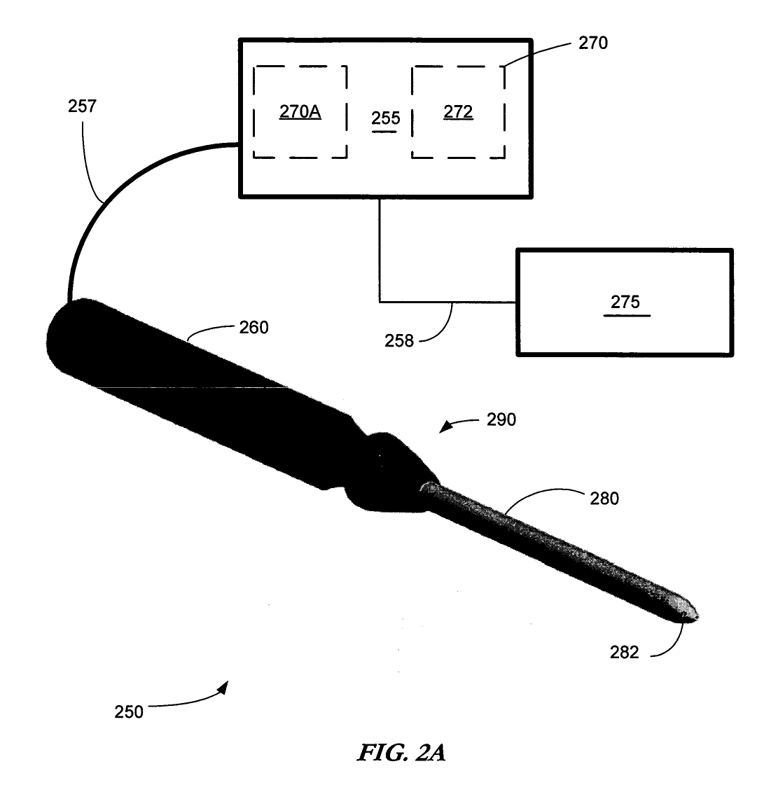

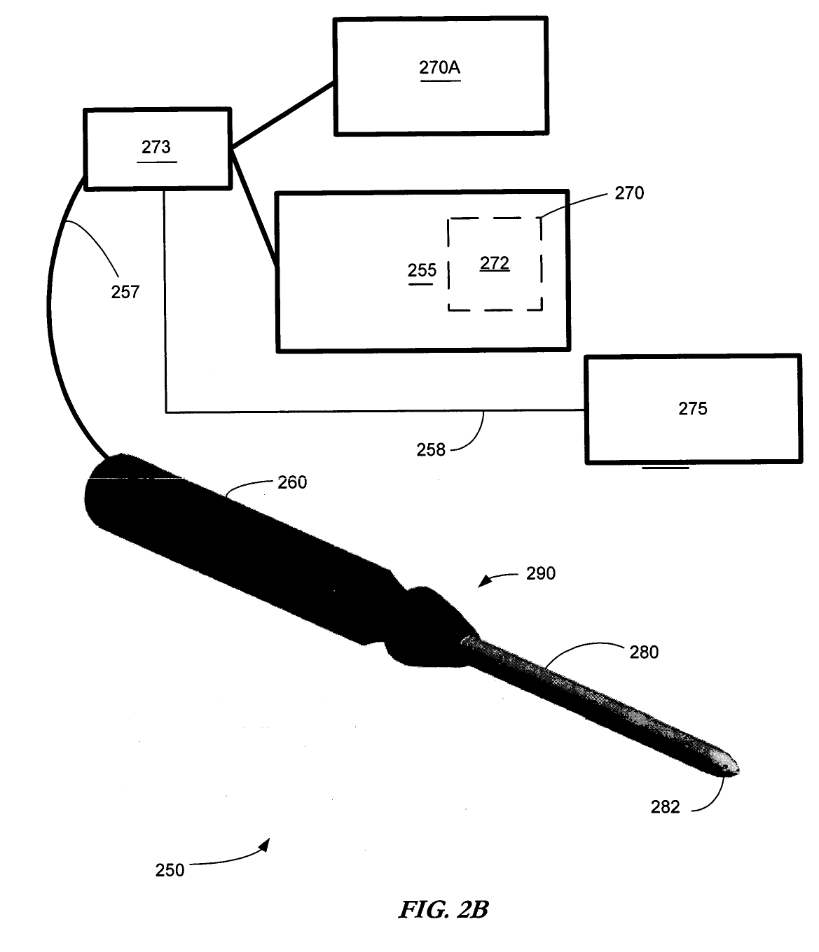

Embodiments of a delivery system according to the present invention are illustrated in FIGS. 2A and 2B. A subcutaneous dissection system 250 includes a subcutaneous dissector 290 including a handle 260 and an elongated dissecting member 280. A fluid, such as a pharmacological agent 272, is stored in a reservoir 270, and may be pumped by a pump 255 through a tubing 257 and delivered to tissue through a port system 282 of the subcutaneous dissector 290. Pump 255 may be turned off and on using a control 275.

It may also be desirable to provide aspiration with the subcutaneous dissection system 250. An aspirant reservoir 270A may optionally be associated with the dissector 290. The aspirant reservoir 270A may be fluidly connected with the pump 255, whereby the pump 255 operates in a first mode to pump fluid into the subcutaneous dissector 290, and operates in a second mode to aspirate aspirant from the subcutaneous dissector 290. The aspirant reservoir 270A may also be connected to a vacuum system or other means of providing aspiration as is known in the art.

FIG. 2B is a perspective view of another subcutaneous dissection system in accordance with the present invention. In FIG. 2B, the aspirant reservoir 270A is connected to the subcutaneous dissector 290 through a valve 273. The pump 255 may also be fluidly connected to the subcutaneous dissector 290 through the valve 273. The valve 273 may be adapted to alternate between aspiration, irrigation, and/or fluid pumping modes. The tubing 257 may be a single lumen tubing, a multiple lumen tubing, or a multiple tube arrangement. The valve 273 may be adapted to provide simultaneous aspiration and pumping through a multiple lumen or multiple tubing arrangement. The valve 273 may be operated via actuation of the control 275.

The control 275 may be connected to the pump 255 or the valve 273 by, for example, wiring 258. The control 275 may be, for example, a switch, a foot pedal, or other actuator capable of controlling the pump 255 and/or the valve 273. The control 275 may be, for example, a switch located on the handle 260, a foot pedal located within reach of a clinician's feet, or implemented within the pump 255 as a voice-activated solenoid actuated valve.

The pump 255 delivers the pharmacological agent 272 through tubing 257. Although the tubing 257 is illustrated as an element separate from the subcutaneous dissector 290, it is contemplated that some or all of the components illustrated in FIGS. 2A and 2B may be enclosed within the subcutaneous dissector 290, for example, within the handle 260. It is also contemplated that the tubing 257 may enter the subcutaneous dissector 290 distal to the handle 260, such as, for example, directly to the elongated dissecting member 280. It is further contemplated that the subcutaneous dissector 290 could be adapted to interface to a robotic surgical system by, for example, adapting the handle 260 to interface with a robotic arm instead of a clinician's hand.

The pharmacological agent 272 may comprise any agent helpful to the efficacy of the subcutaneous dissector 290. The pharmacological agent 272 may comprise, for example, saline solution, phosphated buffer solution, an analgesic, an antibiotic, a hemostatic agent, an anti-inflammatory, or other useful drug or fluid.

For example, a non-exhaustive, non-limiting list of analgesics includes both fast acting and long acting drugs. PROCAINE, for example, can provide fast acting pain relief. BUPIVACAINE, LIDOCAINE, and MAPRIVACAINE, for example, can provide long acting pain relief.

A non-limiting example of a useful antibiotic is VANCOMYCIN, and a non-limiting example of an antiseptic in accordance with the present invention is CEFALOZIN. VANCOMYCIN can be used for the treatment of infection, and CEFALOZIN can be used to prevent possible infection along the dissection path.

A non-exhaustive, non-limiting list of anti-inflammatory drugs includes the glucocorticoid family of drugs (steroids). Useful anti-inflammatory drugs include DEXAMETHASONE, BETAMETHASONE, and IBUPROFIN, for example.

A non-exhaustive, non-limiting list of agents that may improve the electrical properties of dissected tissue includes the glucocorticoid family of drugs, including, for example, DEXAMETHASONE and BETAMETHASONE. These and other candidate drugs may provide for lower chronic defibrillation and pace/sense thresholds for subcutaneous lead/electrode systems. These and other fluids and/or drugs can be delivered individually or in desired combinations prior to, during, and after dissection for purposes of enhancing patient comfort, fighting infections, lowering defibrillation thresholds, and/or chemically treating other conditions.

In FIGS. 2A and 2B, the elongated dissecting member 280 is illustrated as a straight member. However, it is contemplated that the elongated dissection member 280 may have any useful shape. For example, the elongated dissecting member 280 may be curved in one or more planes, and may have a simple or complex curvature defined by one or more radii. The radii of curvature can range from about 25 cm to about 2.5 cm, for example. The elongated dissecting member 280 may be pre-formed in a curved shape, or may be malleable into any shape desired by the clinician.

The elongated dissecting member 280 may, for example, have a pre-defined curvature to properly position an ITCS electrode relative to the can for proper location of the electric field relative to a patients' heart. The elongated dissecting member 280 may also, or alternately, have a pre-defined curvature that can easily follow the curvature of the rib cage for proper dissection. It is contemplated that any combination of predefined shapes with varying levels of malleability can be utilized in the present invention. It is also contemplated that multiple curvatures may also be used. For example, a first curvature in a first direction may help the dissector conform to the curvature of the rib cage, while a second curvature in a second direction may be useful for optimally locating the leads and can relative to the heart or other anatomy. As mentioned above, the curvature of the elongated dissecting member 280 may be defined by a single radius, or by multiple radii or varying radii.

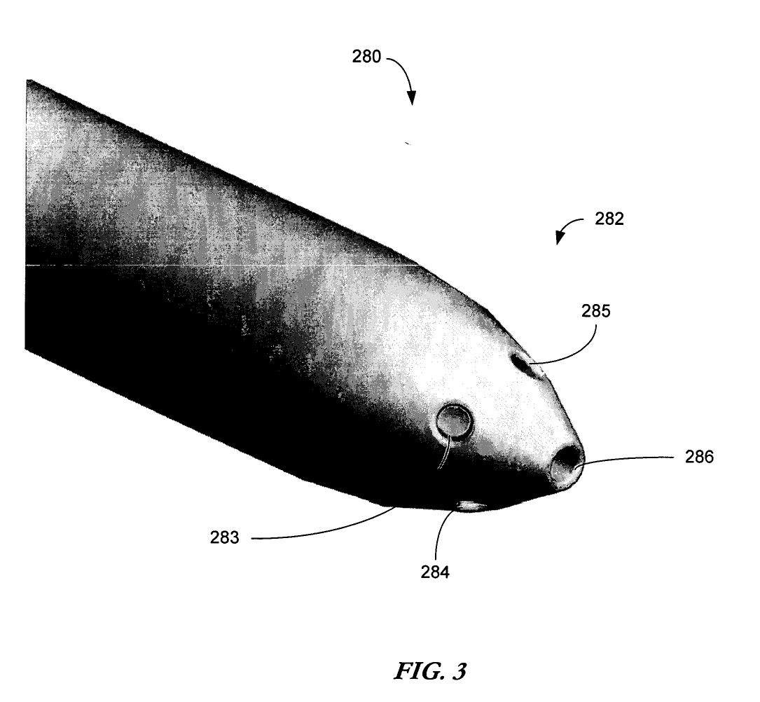

FIG. 3 is a magnified perspective view of the distal end of the elongated dissecting element 280 shown in FIGS. 2A and 2B. A port system 282 is depicted as having an axial aperture 286 and a number of lateral apertures 283, 284, and 285. Depiction of the apertures 283, 284, 285 and 286 is for purposes of clarity of explanation, and not of limitation. It is contemplated that a single aperture, or any number of apertures, may be located on the elongated dissecting element 280 at any location.

For example, a single or series of apertures may be located proximally from the distal end of the elongated dissecting member 280 to provide a pharmacological agent or other fluid anywhere along the path of dissection. If, for example, an analgesic is delivered during dissection, it may be efficacious to provide a number of ports of port system 282 at the distal end of the dissector to ease the pain of dissection, but also to deliver incremental amounts of analgesic along the length of the elongated dissecting member 280 as the dissector progresses into tissue.

The pharmacological agent 272 may be delivered continuously from the port system 282 during dissection. It is also contemplated that the pharmacological agent 272 may be delivered in bolus fashion at time intervals, or only delivered on demand through actuation of the control 275. For example, the pharmacological agent 272 may be delivered when a clinician desires to flush out debris from the dissection path, and may deliver saline solution to remove the debris.

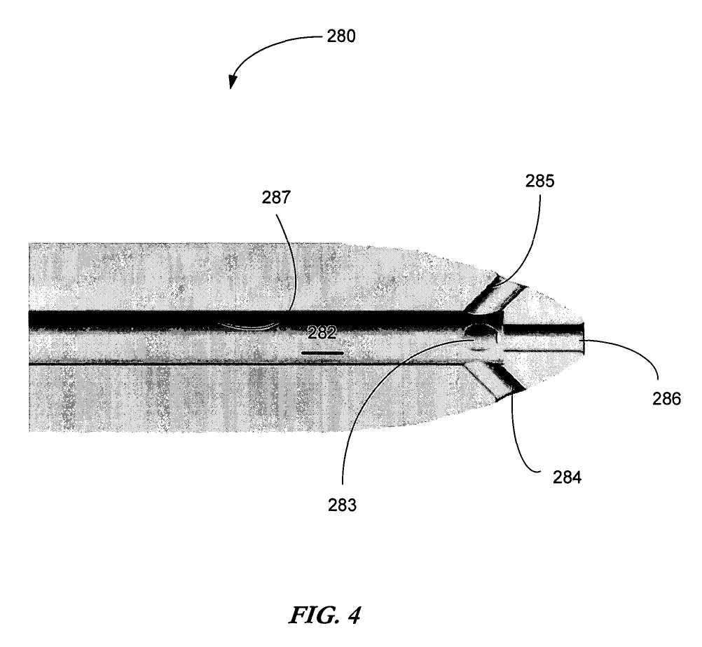

FIG. 4 is a magnified sectional view bisecting the distal end illustrated in FIG. 3. In FIG. 4, the port system 282 is illustrated as including a single channel 287 terminating in the port system 282. Apertures 283, 284, 285, and 286 are fluidly coupled to the channel 287 via branch channels to provide an exit point for a pharmacological or other fluid. The channel 287 may be, for example, molded or machined from plastic or other suitable material. For example, the elongated dissecting element 280 may be injection molded from a suitable material, and include one or more channels 287. The elongated dissecting member 280 may include a number of channels 287 terminating in a number of port systems 282 to provide delivery of a variety of fluids and/or pharmacological agents 272, and/or to provide delivery of fluids and/or pharmacological agents 272 to different locations along the length of the elongated dissecting member 280.

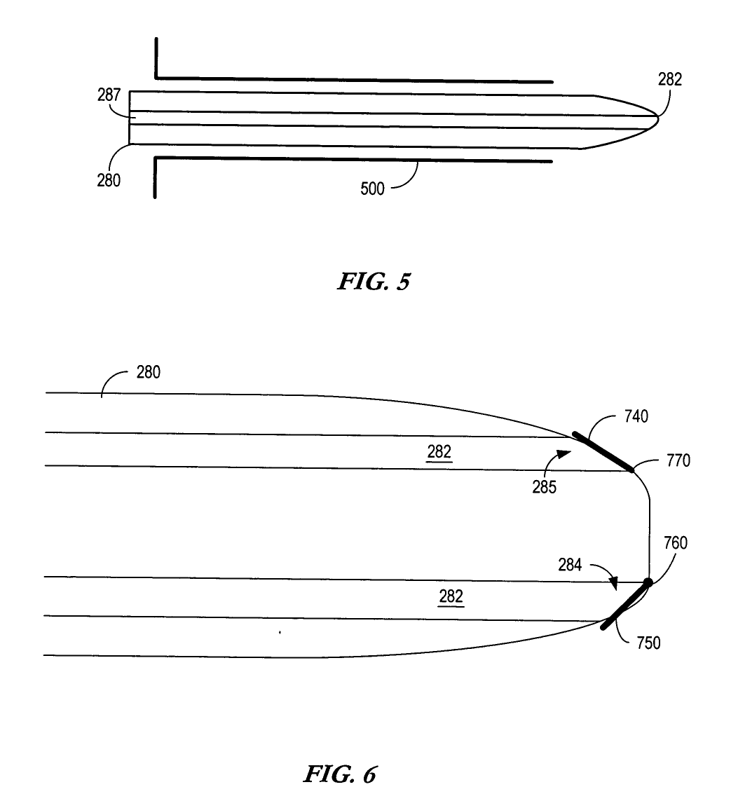

Referring now to FIG. 5, a sheath 500 may be provided to surround the elongated dissecting member 280. The sheath 500 may extend along a portion of, or the entire length of, the elongated dissecting member 280. As dissection occurs, the sheath 500 may be inserted along the dissection path. Alternatively, the sheath 500 may be inserted after the dissection procedure is completed. Upon completion of the dissection procedure, the subcutaneous dissector 290 can be removed from the patient's body. After removal of the subcutaneous dissector 290, the sheath 500 may be left in-place, to provide a guide for placement of electrodes and/or the ITCS housing. After placement of the electrodes and/or housing, the sheath 500 may be stripped out, leaving the electrodes properly positioned.

In one embodiment, the sheath 500 may include one or more longitudinal pre-stress lines extending between a distal end and a proximal end of the sheath 500. The sheath 500 can optionally include a sheath handle. The pre-stress line provides for a peel-away or tear-away sheath 500 that facilitates extraction of the sheath 500 from the body. If the sheath 500 is provided with a sheath handle, the sheath handle is preferably separable into at least two sections such that sheath handle separation splits the sheath along the longitudinal pre-stress line at the proximal end of the sheath 500. The sheath 500 (with or without a sheath handle) splits along the longitudinal pre-stress line upon sheath retraction in a proximal direction.

FIG. 6 is a plan view of the distal end of the elongated dissecting member 280 including the port system 282 having apertures 284 and 285. In this embodiment, the elongated dissection member 280 further includes a aperture cover or valve 740 shown covering the aperture 284. The aperture cover 740 may be attached to the elongated dissecting member 280 by, for example, a hinge 770. The hinge 770 may be a living hinge or other hinge known in the art. A second aperture cover 750 is illustrated covering the aperture 285.

The aperture covers 740, 750 may be useful to prevent clogging of the port system 282 during tissue dissection. More particularly, the aperture covers 740 and 750 may be used to advantageously limit entry of dissected tissue or other debris into the port system 282 as the elongated dissecting member 280 is advanced into tissue. Only when the pressure of the fluid within the channel 287 exceeds the pressure exerted by the surrounding tissue on the aperture cover 750 would the fluid be delivered from the port system 282.

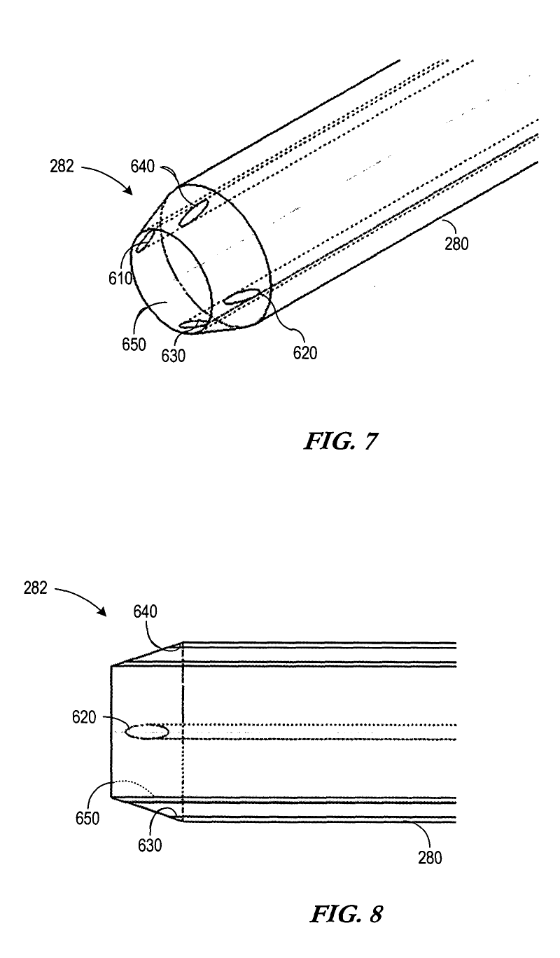

FIGS. 7 and 8 illustrate a perspective and plan view respectively of another embodiment of the elongated dissecting member 280 incorporating a port system 282 in accordance with the present invention. A lead lumen 650 is illustrated as a central lumen of the elongated dissecting member 280. The lead lumen 650 may be used to deliver leads into dissected tissue space. The lead lumen 650 is shown surrounded by fluid lumens 610, 620, 630, and 640. The lead lumen 650 is configured to receive and permit passage of a lead, such as a pacing lead, a defibrillation lead or a sensor lead.

In the embodiment depicted in FIGS. 7 and 8, the fluid lumens 610, 620, 630, and 640 are fluidly independent from one another. The fluid lumens 610, 620, 630, and 640 may be used, for example, to deliver a first pharmacological agent from fluid lumen 610, a second pharmacological agent from fluid lumen 620, irrigation fluid from fluid lumen 630 and provide aspiration from fluid lumen 640. It is understood that various types of fluids (e.g., fluid comprising a single agent or combination of agents) may be delivered concurrently, or in an independently controlled manner, through selected fluid lumens 610, 620, 630, and 640. For example, a single type of fluid may be delivered concurrently, or in an independently controlled manner, through all or selected fluid lumens 610, 620, 630, and 640.

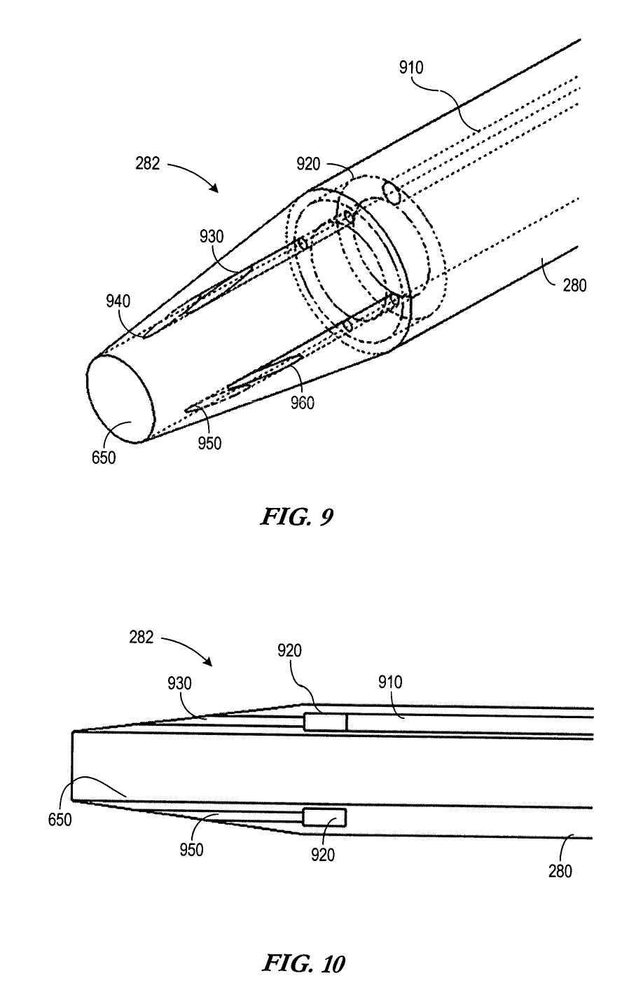

FIGS. 9 and 10 illustrate perspective and plan views, respectively, of yet another embodiment of the elongated dissecting member 280 that incorporates a port system 282 in accordance with the present invention. A lead lumen 650 is illustrated as a central lumen of the elongated dissecting member 280, here surrounded by fluid lumens 930, 940, 950, and 960. The fluid lumens 930, 940, 950, and 960 extend from a manifold 920 to the distal end of the elongated dissecting member 280.

The manifold 920 may receive a fluid, such as a fluid comprising a pharmacological agent, from a channel system 910, and distribute the fluid to each of the fluid lumens 930, 940, 950, and 960. In the case of aspiration, fluid and debris entering the fluid lumens 930, 940, 950, and 960 can be transported to the proximal end of the elongated dissecting member 280 via the manifold 920 and the channel system 910 and collected externally of the dissector. This configuration provides for distributed delivery of a pharmacological agent and/or irrigation fluids to tissue as the tissue is subject to dissection.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A dissection tool, comprising:
a handle having a proximal end and a distal end;
an elongated dissecting member having a proximal end and a distal end, the elongated dissecting member extending from the distal end of the handle; and
a fluid channel system extending from at least the proximal end of the elongated dissecting member to the distal end of the elongated dissecting member, the fluid channel system configured for one or both of fluid transport and aspiration between at least the proximal and distal ends of the elongated dissecting member.

2. The dissection tool of claim 1, wherein the fluid channel system comprises a port system, the port system comprising a plurality of apertures.

3. The dissection tool of claim 1, wherein the fluid channel system comprises a port system, the port system comprising at least one aperture and at least one aperture cover, the aperture cover hingedly mounted at the distal end of the elongated dissecting member and moveable between a closed orientation and an open orientation relative to the aperture.

4. The dissection tool of claim 1, wherein the fluid channel system comprises a port system and a manifold disposed between the fluid channel system and the port system, the manifold adapted to distribute a fluid between the fluid channel system and the port system.

5. The dissection tool of claim 1, wherein the fluid channel system comprises a plurality of channels terminating in a plurality of apertures, wherein each channel terminates in a single opening.

6. The dissection tool of claim 1, wherein the fluid channel system comprises a plurality of channels terminating in a plurality of apertures, wherein at least one of the channels terminates in a second plurality of apertures.

7. The dissection tool of claim 1, wherein the elongated dissecting member is curved.

8. The dissection tool of claim 7, wherein the curvature of the elongated dissecting member is defined by a single radii.

9. The dissection tool of claim 7, wherein the curvature of the elongated dissecting member is defined by multiple radii.

10. The dissection tool of claim 7, wherein the curvature of the elongated dissecting member has a radius of curvature ranging from about 25 centimeters to about 2.5 centimeters.

11. The dissection tool of claim 1, wherein the fluid channel system is adapted to transport an irrigation fluid.

12. The dissection tool of claim 1, wherein the fluid delivery system is adapted to transport a pharmacological agent.

13. The dissection tool of claim 1, wherein the fluid channel system comprises a first fluid channel and a second fluid channel, the first fluid channel adapted to transport an irrigation fluid and the second fluid channel adapted to transport a pharmacological agent.

14. The dissection tool of claim 1, wherein the fluid channel system is adapted to transport an analgesic or an anesthetic.

15. The dissection tool of claim 1, wherein the fluid channel system is adapted to transport an antibiotic or an antiseptic.

16. The dissection tool of claim 1, wherein the fluid channel system is adapted to transport an anti-inflammatory agent.

17. The dissection tool of claim 1, wherein the fluid channel system is adapted to transport a fluid comprising an agent that improves hemostasis or an agent that accelerates healing.

18. The dissection tool of claim 1, wherein the fluid channel system is adapted to transport a fluid comprising an agent that enhances electrical properties of tissue.

19. A dissection system, comprising:
a pump;
a fluid reservoir adapted to provide a fluid to the pump;
a tube; and
a dissection tool, comprising:
a handle having a proximal end and a distal end;
an elongated dissecting member having a proximal end and a distal end, the elongated dissecting member extending from the distal end of the handle; and
a fluid channel system extending from at least the proximal end of the elongated dissecting member to the distal end of the elongated dissecting member, wherein the tube fluidly connects the dissection tool with the pump.

20. The dissection system of claim 19, wherein the fluid channel system comprises a port system, the port system comprising a plurality of apertures.

21. The dissection system of claim 20, further comprising a manifold disposed between the fluid channel system and the port system, the manifold adapted to distribute fluid between the fluid channel system and the port system.

22. The dissection system of claim 19, wherein the port system comprises at least one aperture and at least one aperture cover, the aperture cover hingedly mounted to the distal end of the elongated dissecting member and moveable between a closed orientation and an open orientation relative to the aperture.

23. The dissection system of claim 22, wherein the aperture cover is movable from the closed orientation to the open orientation in response to delivery of the fluid through the fluid channel system at a pressure that exceeds a pressure exerted on the aperture cover by surrounding tissue.

24. The dissection system of claim 19, wherein the fluid channel system comprises a plurality of channels each terminating in a single aperture or a plurality of apertures.

25. The dissection system of claim 19, wherein the pump comprises a pump controller actuatable by a clinician to control dispensing of the fluid from the fluid reservoir to the dissection tool.

26. The dissection system of claim 19, wherein the fluid channel system comprises a first fluid channel and a second fluid channel, the first fluid channel adapted to transport an irrigation fluid and the second fluid channel adapted to transport a pharmacological agent.

27. The dissection system of claim 19, further comprising a sheath removably displaceable over the elongated dissecting member.

28. The dissection system of claim 19, wherein the fluid comprises an irrigation fluid.

29. The dissection system of claim 19, wherein the fluid comprises a pharmacological agent.

30. The dissection system of claim 19, wherein the fluid comprises an analgesic or an anesthetic.

31. The dissection system of claim 19, wherein the fluid comprises an antibiotic or an antiseptic.

32. The dissection system of claim 19, wherein the fluid comprises an anti-inflammatory agent.

33. A method of dissecting subcutaneous tissue, comprising:
providing a dissection tool having a fluid delivery system;
dissecting subcutaneous tissue with the dissection tool; and
delivering a fluid from the dissection tool during subcutaneous tissue dissection via the fluid delivery system.

34. The method of claim 33, wherein the dissection is performed only in a subcutaneous tissue plane.

35. The method of claim 33, wherein a plane of dissection follows a curvature of the rib cage.

36. The method of claim 33, wherein the dissection tool further comprises a curved elongated dissecting member.

37. The method of claim 33, wherein delivering the fluid comprises dispensing the fluid from a single dissection tool location.

38. The method of claim 33, wherein delivering the fluid comprises dispensing the fluid from multiple dissection tool locations.

39. The method of claim 33, further comprising limiting entry of dissected tissue or other debris into the fluid delivery system as the dissection tool is advanced through the subcutaneous tissue.

40. The method of claim 33, further comprising aspirating fluids or other debris from the fluid delivery system.

41. The method of claim 33, wherein delivering the fluid comprises delivering an irrigation fluid.

42. The method of claim 33, wherein delivering the fluid comprises delivering a pharmacological agent.

43. The method of claim 33, wherein delivering the fluid comprises delivering a gas.

44. The method of claim 33, wherein delivering the fluid comprises delivering an analgesic or an anesthetic.

45. The method of claim 33, wherein delivering the fluid comprises delivering an antibiotic or an antiseptic.

46. The method of claim 33, wherein delivering the fluid comprises delivering an anti-inflammatory agent.

47. A method of implanting subcutaneous electrodes, comprising:
providing a dissection tool having a fluid delivery channel;
dissecting subcutaneous tissue with the dissection tool to create a path in the subcutaneous tissue;
delivering a fluid from the dissection tool during subcutaneous tissue dissection; and
delivering one or more electrodes into the subcutaneous tissue path.

48. The method of claim 47, wherein the dissection is performed only in a subcutaneous tissue plane.

49. The method of claim 47, wherein a plane of dissection follows a curvature of the rib cage.

50. The method of claim 47, further comprising delivering a housing into the subcutaneous tissue path, wherein the housing matingly connects with the one or more electrodes.

51. The method of claim 47, further comprising advancing a sheath into the subcutaneous tissue path.

52. The method of claim 51, wherein the sheath is advanced into the subcutaneous tissue path during subcutaneous tissue dissection.

53. The method of claim 51, further comprising delivering the one or more electrodes into the subcutaneous tissue path via the sheath.

54. The method of claim 51, wherein the dissection tool further comprises a lead lumen, the method further comprising delivering a lead to a subcutaneous body location via the lead lumen of the dissection tool.

55. The method of claim 51, further comprising aspirating fluid or debris through the fluid delivery channel or through a second fluid delivery channel of the dissection tool fluidly isolated from the fluid delivery channel.

56. The method of claim 47, wherein delivering the fluid comprises dispensing the fluid from a single dissection tool location or from multiple dissection tool locations.

57. The method of claim 47, further comprising limiting entry of dissected tissue or other debris into the fluid delivery system as the dissection tool is advanced through the subcutaneous tissue.

58. The method of claim 47, wherein delivering the fluid comprises delivering an irrigation fluid.

59. The method of claim 47, wherein delivering the fluid comprises delivering a pharmacological agent.

60. The method of claim 47, wherein the fluid delivery channel is adapted to transport an analgesic or an anesthetic.

61. The method of claim 47, wherein the fluid delivery channel is adapted to transport an antibiotic or an antiseptic.

62. The method of claim 47, wherein the fluid delivery channel is adapted to transport an anti-inflammatory agent.

63. A dissection tool, comprising:
a handle having a proximal end and a distal end;
means, attached to the distal end of the handle, for dissecting subcutaneous tissue; and
means for delivering a fluid through or along the dissecting means and to tissue subject to dissection.

64. The dissection tool of claim 63, further comprising means for limiting entry of dissected tissue or other debris into the fluid delivery means as the dissection tool is advanced through the subcutaneous tissue.

65. The dissection tool of claim 63, wherein the dissecting means comprises one or more curved portions.

66. The dissection tool of claim 63, wherein the fluid delivering means comprises a fluid distribution manifold.

67. The dissection tool of claim 63, further comprising means for controllably dispensing the fluid to the fluid delivering means.

68. The dissection tool of claim 63, wherein the delivering means is adapted to transport an analgesic or an anesthetic.

69. The dissection tool of claim 63, wherein the delivering means is adapted to transport an antibiotic or an antiseptic.

70. The dissection tool of claim 63, wherein the delivering means is adapted to transport an anti-inflammatory agent.

71. A kit, comprising:
at least one subcutaneous dissection tool, the dissection tool comprising a fluid channel system; and
at least one subcutaneous electrode configured to be received by a subcutaneous tissue tunnel producible by the at least one subcutaneous dissection tool.

72. The kit according to claim 71, further comprising a fluid delivery system configured for coupling to the at least one dissection tool.

73. The kit according to claim 71, comprising an assortment of subcutaneous dissection tools having varying configurations.

74. The kit according to claim 71, comprising an assortment of subcutaneous dissection tools having elongated dissecting members of varying curvature.

75. The kit according to claim 71, wherein the dissection tool comprises a handle section configured to accommodate a plurality of separable elongated dissecting members.

76. The kit according to claim 71, further comprising a sheath configured to be removably displaceable over an elongated dissecting member of the dissection tool.

77. The kit according to claim 71, further comprising an implantable medical device, a housing of the implantable medical device configured to be received by the subcutaneous tissue tunnel, the housing configured for coupling to the at least one subcutaneous electrode.

78. The kit according to claim 71, wherein the fluid channel system is adapted to transport an analgesic or an anesthetic.

79. The kit according to claim 71, wherein the fluid channel system is adapted to transport an antibiotic or an antiseptic.

80. The kit according to claim 71, wherein the fluid channel system is adapted to transport an anti-inflammatory agent.

81. A dissection tool, comprising:
a handle having a proximal end and a distal end;
an elongated dissecting member extending from the distal end of the handle, the elongated dissecting member comprising:
a proximal end;
a distal end; and
a lead lumen extending between the proximal and distal ends; and
a fluid channel system extending between at least the proximal end of the elongated dissecting member and the distal end of the elongated dissecting member.

82. The dissection tool of claim 81, wherein the lead lumen is configured to receive a pacing lead.

83. The dissection tool of claim 81, wherein the lead lumen is configured to receive a defibrillator lead.

84. The dissection tool of claim 81, wherein the lead lumen is configured to receive a sensor lead.

85. The dissection tool of claim 81, wherein the fluid channel system further comprises a port system having a plurality of apertures.

86. The dissection system of claim 85, wherein the port system comprises a manifold, the manifold adapted to distribute the fluid between the fluid channel system and the plurality of apertures.

87. The dissection tool of claim 85, wherein the port system comprises at least one aperture and at least one aperture cover, the aperture cover hingedly mounted to the distal end of the elongated dissecting member and moveable between a closed orientation and an open orientation relative to the aperture.

88. The dissection tool of claim 81, wherein the fluid channel system comprises a plurality of channels terminating in a plurality of apertures, wherein each channel terminates in a single opening.

89. The dissection tool of claim 81, wherein the fluid channel system comprises a plurality of channels terminating in a plurality of apertures, wherein at least one of the channels terminates in a second plurality of apertures.

90. The dissection tool of claim 81, wherein the elongated dissecting member is curved.

91. The dissection tool of claim 81, wherein the fluid channel system is adapted to transport an irrigation fluid.

92. The dissection tool of claim 81, wherein the fluid delivery system is adapted to transport a pharmacological agent.

93. The dissection tool of claim 81, wherein the fluid delivery system is adapted for fluid transport and aspiration.

94. The dissection tool of claim 81, wherein the fluid channel system is adapted to transport an analgesic or an anesthetic.

95. The dissection tool of claim 81, wherein the fluid channel system is adapted to transport an antibiotic or an antiseptic.

96. The dissection tool of claim 81, wherein the fluid channel system is adapted to transport an anti-inflammatory agent.

97. A dissection system, comprising:
a pump;
a fluid reservoir adapted to provide a fluid to the pump;
an aspiration reservoir adapted to contain aspirant;
a tube; and
a dissection tool, comprising:
a handle having a proximal end and a distal end;
an elongated dissecting member having a proximal end and a distal end, the elongated dissecting member extending from the distal end of the handle; and
a fluid channel system extending from at least the proximal end of the elongated dissecting member to the distal end of the elongated dissecting member, wherein the tube fluidly connects the dissection tool with the fluid reservoir and the aspiration reservoir.

98. The dissection system of claim 97, wherein the fluid channel system comprises a port system, the port system comprising a plurality of apertures.

99. The dissection system of claim 97, further comprising a valve fluidly coupled to the fluid reservoir and the dissection tool, the valve adapted to fluidly couple the fluid channel system with a selected one of the fluid reservoir and the aspirant reservoir.

100. The dissection system of claim 97, wherein the pump comprises a pump controller actuatable by a clinician to control dispensing of the fluid from the fluid reservoir to the dissection tool or the aspiration of aspirant into the aspirant reservoir.

101. The dissection system of claim 97, wherein the fluid channel system comprises a first fluid channel and a second fluid channel, the first fluid channel adapted to transport an irrigation fluid and the second fluid channel adapted to transport a pharmacological agent.

102. The dissection system of claim 97, wherein the fluid comprises an irrigation fluid.

103. The dissection system of claim 97, wherein the fluid comprises a pharmacological agent.

104. The dissection system of claim 97, wherein the fluid comprises an analgesic or an anesthetic.

105. The dissection system of claim 97, wherein the fluid comprises an antibiotic or an antiseptic.

106. The dissection system of claim 97, wherein the fluid comprises an anti-inflammatory agent.
